SEQUENCE LISTING (10 sequences)

SEQ ID NO: 1 - protein, 121 aa, Artificial Sequence [sequence omitted]

SEQ ID NO: 2 - protein, 108 aa, Artificial Sequence
  Light Chain Variable Domain
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKR

SEQ ID NO: 3 - protein, 7 aa, Artificial Sequence
  HVR-H1
LSGDWIH

SEQ ID NO: 4 - protein, 12 aa, Artificial Sequence
  HVR-H2
VGEISAAGGYTD

SEQ ID NO: 5 - protein, 14 aa, Artificial Sequence
  HVR-H3
ARESRVSFEAAMDY

SEQ ID NO: 6 - protein, 7 aa, Artificial Sequence
  HVR-L1
NIATDVA

SEQ ID NO: 7 - protein, 4 aa, Artificial Sequence
  HVR-L2
SASF

SEQ ID NO: 8 - protein, 7 aa, Artificial Sequence
  HVR-L3
SEPEPYT

SEQ ID NO: 9 - protein, 451 aa, Artificial Sequence
  Heavy Chain
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Light Chain
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2014/0271634 A1
(43) Pub. Date: Sep. 18, 2014

(54) COMBINATIONS OF A MEK INHIBITOR COMPOUND WITH AN HER3/EGFR INHIBITOR COMPOUND AND METHODS OF USE

(71) Applicants: The Regents of the University of California, Oakland, CA (US); Genentech, Inc., South San Francisco, CA (US)

(72) Inventors: Mark X. Sliwkowski, San Carlos, CA (US); Wolfgang Michael Korn, Kentfield, CA (US)

(73) Assignees: The Regents of the University of California, Oakland, CA (US); Genentech, Inc., South San Francisco, CA (US)

(21) Appl. No.: 14/210,608

(22) Filed: Mar. 14, 2014

Related U.S. Application Data
(60) Provisional application No. 61/782,734, filed on Mar. 14, 2013.
(60) Provisional application No. 61/902,870, filed on Nov. 12, 2013.

(51) Int. Cl. A61K 39/395; A61K 31/437; A61K 31/4523

(52) U.S. Cl. CPC A61K 39/39558; A61K 31/4523; A61K 31/437
USPC 424/133.1

(57) ABSTRACT

The invention provides combinations comprising a MEK inhibitor (such as GDC-0973 or GDC-0623), or a pharmaceutically acceptable salt thereof and a HER3/EGFR inhibitor (such as MEHD7945A). The combinations are particularly useful for treating hyperproliferative disorders, such as cancer.

### CROSS REFERENCE TO RELATED APPLICATIONS

This application claims the benefit of U.S. Provisional Application No. 61/782,734, filed on Mar. 14, 2013, and claims the benefit of U.S. Provisional Application No. 61/902,870, filed on Nov. 12, 2013, the disclosures of which are incorporated herein by reference in their entirety.

### SEQUENCE LISTING

The present application contains a Sequence Listing which has been submitted in ASCII format via EFS-Web and is hereby incorporated by reference in its entirety. Said ASCII copy, created on Mar. 11, 2104, is named P5587R1US_ST25.txt and is 9,498 bytes in size.

### FIELD OF THE INVENTION

The invention relates generally to pharmaceutical combinations of compounds with activity against hyperproliferative disorders such as cancer that include a combination of a compound that inhibits the MEK pathway with a compound that blocks HER3/EGFR. The invention also relates to methods of using the combinations for in vitro, in situ, and in vivo diagnosis or treatment of mammalian cells, or associated pathological conditions.

### BACKGROUND OF THE INVENTION

Protein kinases (PK) are enzymes that catalyze the phosphorylation of hydroxy groups on tyrosine, serine and threonine residues of proteins by transfer of the terminal (gamma) phosphate from ATP. Through signal transduction pathways, these enzymes modulate cell growth, differentiation and proliferation, i.e., virtually all aspects of cell life in one way or another depend on PK activity (Hardie, G. and Hanks, S. (1995) The Protein Kinase Facts Book. I and II, Academic Press, San Diego, Calif.). Furthermore, abnormal PK activity has been related to a host of disorders, ranging from relatively non-life threatening diseases such as psoriasis to extremely virulent diseases such as glioblastoma (brain cancer). Protein kinases are an important target class for therapeutic modulation (Cohen, P. (2002) Nature Rev. Drug Discovery 1:309).

MEK is a dual-specificity kinase that phosphorylates tyrosines and threonines required for activation on ERK 1 and 2. Two related genes encode MEK1 and MEK2 which differ in their binding to ERKs. HER3 a receptor tyrosine kinase that can be bound and activated by neuregulins and NTAK. EGFR is a transmembrane glycoprotein that is a receptor for members of the epidermal growth factor family.

Currently, there remains a need for improved methods and compositions that can be used to treat hyperproliferative diseases such as cancer.

### SUMMARY OF THE INVENTION

It has been determined that improved effects in inhibiting the growth of cancer cells in vitro and in vivo can be achieved by inhibiting MEK, HER3 and EGFR. It has been found, for example, that improved effects in inhibiting the growth of cancer cells in vitro and in vivo can be achieved by administering a combination of GDC-0973 or GDC-0623, or a pharmaceutically acceptable salt thereof, and MEHD7945A, for the therapeutic treatment of a hyperproliferative disorder. The combinations and methods will be useful in the treatment of hyperproliferative disorders such as cancer. In certain embodiments, administration of the combinations may provide synergistic effects.

Accordingly, certain embodiments of the invention provide therapeutic combinations comprising the small-molecule MEK inhibitor GDC-0973 (Formula I), or a pharmaceutically acceptable salt thereof (see WO 2007/044515) and having the structure:

[chemical structure]

or the small-molecule MEK inhibitor GDC-0623 (Formula II), or a pharmaceutically acceptable salt thereof (see WO2009/085983), and having the structure:

[chemical structure]

in combination with MEHD7945A, a dual-action antibody which comprises two identical antigen binding domains, each of which specifically binds to both HER3 and EGFR (see DL11f in WO 2010/108127 (e.g., FIG. 33) and Schaefer et al., Cancer Cell, 20, 472-486 (2011)). MEHD7945A and GDC-0973 or GDC-0623 may be present in two separate pharmaceutical compositions or together in a single pharmaceutical composition.

Accordingly, certain embodiments of the invention are directed to a combination of GDC-0973 or GDC-0623, or a pharmaceutically acceptable salt thereof and MEHD7945A, for the therapeutic treatment of a hyperproliferative disorder.

In certain embodiments, the hyperproliferative disorder is cancer.

In certain embodiments, the cancer is associated with the KRAS mutation.

In certain embodiments, the cancer is selected from, colorectal, mesothelioma, endometrial, pancreatic, breast, lung, ovarian, prostate, melanoma, gastric, colon, renal, head and neck, and glioblastoma.

In certain embodiments GDC-0973 or a pharmaceutically acceptable salt thereof is administered in combination with MEHD7945A.

In certain embodiments, GDC-0623 or a pharmaceutically acceptable salt thereof is administered in combination with MEHD7945A.

In certain embodiments, GDC-0973 or GDC-0623, or a pharmaceutically acceptable salt thereof is administered simultaneously with MEHD7945A.

In certain embodiments, GDC-0973 or GDC-0623, or a pharmaceutically acceptable salt thereof and MEHD7945A are administered sequentially.

Certain embodiments of the invention are directed to a combination of GDC-0973 or GDC-0623, or a pharmaceutically acceptable salt thereof and MEHD7945A for therapeutic use for improving the quality of life of a patient having a hyperproliferative disorder.

Certain embodiments of the invention are directed to a combination of GDC-0973 or GDC-0623, or a pharmaceutically acceptable salt thereof; and MEHD7945A, for treating a hyperproliferative disorder.

Certain embodiments of the invention are directed to a use of a combination of GDC-0973 or GDC-0623, or a pharmaceutically acceptable salt thereof; and MEHD7945A, in the preparation of a medicament for the treatment of a hyperproliferative disorder in a patient.

Certain embodiments of the invention are directed to a kit comprising GDC-0973 or GDC-0623, or a pharmaceutically acceptable salt thereof; and MEHD7945A, a container, and a package insert or label indicating the administration GDC-0973 and GDC-0623, or a pharmaceutically acceptable salt thereof; and MEHD7945A, for treating a hyperproliferative disorder.

Certain embodiments of the invention are directed to a product comprising GDC-0973 or GDC-0623, or a pharmaceutically acceptable salt thereof and MEHD7945A as a combined preparation for separate, simultaneous or sequential use in the treatment of a hyperproliferative disorder (e.g., cancer).

Certain embodiments of the invention are directed to a method for treating a hyperproliferative disorder in a patient (e.g., cancer), comprising administering to the patient a combination of GDC-0973 and GDC-0623, or a pharmaceutically acceptable salt thereof; and MEHD7945A.

### BRIEF DESCRIPTION OF THE DRAWINGS

FIG. 1 is a graph demonstrating that MEHD7945A binds to both HER3-ECD and EGFR-ECD.

FIGS. 2A and B are graphs demonstrating that MEHD7945A inhibits EGFR and HER2/HER3-dependent signaling.

FIG. 3 is a graph showing inhibition of tumor growth in FaDu cancer model by MEHD7945A.

FIG. 4 is a summary of the tumor growth inhibitory effect of MEHD7945A compared to cetuximab or anti-HER3 in numerous murine xenograft models.

FIG. 5 is a graph demonstrating that GDC-0973 and GDC-0623 are effective in inhibiting the growth of B-RAF mutant tumor cells.

FIG. 6 is a graph demonstrating that GDC-0973 and GDC-0623 are effective in inhibiting the growth of KRAS mutant tumor cells.

FIG. 7 is a graph demonstrating the effect of single agent and combination treatment on pAkt and pERK levels in a murine xenograft CRC KRAS DLD-1 (A) and LS180 (B) models.

FIG. 8 is a graph demonstrating the tumor growth inhibitory effect of single agent and combination treatments of MEHD7945A, GDC-0973 and GDC-0623.

FIG. 9 demonstrates that TGFα-stimulated LS180 or DLD-1 cells treated with cobimetinib showed increased phosphorylation of AKT.

FIG. 10 is a graph demonstrating the inhibition of KRAS-mutant cell line, LS180, proliferation by MEHD7945A and cobimetinib combination.

FIG. 11A is a graph demonstrating the effect of cobimetinib in combination with MEHD7945A on LS180 Colorectal Adenocarcinoma Tumor Xenografts in CD-1 Nude Mice; FIG. 11B is a table summarizing the data from FIG. 11A.

FIG. 12A is a graph demonstrating the effect of cobimetinib in combination with MEHD7945A on KRAS-Mutant DLD-1 Colorectal Adenocarcinoma Tumor Xenografts in C.B-17 SCID beige mice; FIG. 12B is a table summarizing the data from FIG. 12A.

FIG. 13A is a graph demonstrating the effect of cobimetinib in combination with MEHD7945A on BxPC3 Ductal Pancreatic Xenograft Tumors in NCr Nude Mice; FIG. 13B is a table summarizing the anti-tumor activity for this study; FIG. 13C is a table summarizing the Time to Tumor Progression and Response for this study.

### DETAILED DESCRIPTION OF EXEMPLARY EMBODIMENTS

### I. Definitions

It must be noted that as used herein and in the appended claims, the singular forms “a”, “and”, and “the” include plural referents unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

The term “antibody” herein is used in the broadest sense and specifically covers monoclonal antibodies, polyclonal antibodies, multispecific antibodies, and antibody fragments so long as they exhibit the desired biological activity. The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). One specific example of an antigen-binding domain is a V_{H}V_{L} unit comprised of a heavy chain variable domain (V_{H}) and a light chain variable domain (V_{L}). Such multispecific antibodies include, but are not limited to, full length antibodies, antibodies having two or more V_{L} and V_{H} domains, antibody fragments such as Fab, Fv, dsFv, scFv, diabodies, bispecific diabodies and triabodies, antibody fragments that have been linked covalently or non-covalently. A “bispecific antibody” is a multispecific antibody comprising an antigen-binding domain that is capable of specifically binding to two different epitopes on one biological molecule or is capable of specifically binding to epitopes on two different biological molecules. The bispecific antibody is also referred to herein as having “dual specificity” or as being “dual specific”.

In certain embodiments, an antibody of the invention has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M) for its target HER or HERs.

The basic 4-chain antibody unit is a heterotetrameric glycoprotein composed of two identical light (L) chains and two identical heavy (H) chains (an IgM antibody consists of 5 of the basic heterotetramer units along with an additional polypeptide called J chain, and therefore contains 10 antigen-binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has, at the N-terminus, a variable domain (V_{H}) followed by three constant domains (C_{H}) for each of the α and γ chains and four C_{H} domains for μ and ε isotypes. Each L chain has, at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen-binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called hypervariable regions” or HVR. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region,” “HVR,” or “HV,” when used herein refers to the regions of an antibody variable domain which are hypervariable in sequence and/or form structurally defined loops. Generally, antibodies comprise six HVRs; three in the VH (HVR-H1, HVR-H2, HVR-H3), and three in the VL (HVR-L1, HVR-L2, HVR-L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

HVRs generally comprise amino acid residues from the hypervariable loops and/or from the “complementarity determining regions” (CDRs), the latter being of highest sequence variability and/or involved in antigen recognition. A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 47-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see WO 2006/073941).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

An “affinity matured” antibody is one with one or more alterations in one or more HVRs or framework region thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced using certain procedures known in the art. For example, Marks et al. Bio/Technology 10:779-783 (1992) describes affinity maturation by VH and VL domain shuffling. Random mutagenesis of HVR and/or framework residues is described by, for example, Barbas et al. Proc Nat. Acad. Sci. USA 91:3809-3813 (1994); Schier et al. Gene 169:147-155 (1995); Yelton et al., J. Immunol. 155:1994-2004 (1995); Jackson et al., J. Immunol. 154(7):3310-9 (1995); and Hawkins et al, J. Mol. Biol. 226:889-896 (1992).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG₂, IgG₃, IgG₄, IgA1, and IgA₂. The heavy chain constant domains that correspond to the different classes of immunoglobulins are called α, δ, ε, γ, and μ, respectively.

The term “patient” (interchangeably termed “individual” and “subject”) is a human patient. The patient may be a “cancer patient”, i.e. one who is suffering or at risk for suffering from one or more symptoms of cancer.

The terms “treat” and “treatment” refer to therapeutic treatment, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder, e.g., a patient with cancer.

The phrase “therapeutically effective amount” means an amount that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount may reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. To the extent the combination may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

A “chemotherapeutic agent” is a biological (e.g., large molecule) or chemical (e.g., small molecule) compound useful in the treatment of cancer, regardless of mechanism of action.

A “platinum agent” is a chemotherapeutic agent that comprises platinum, for example carboplatin, cisplatin, and oxaliplatin.

The term “mammal” includes, but is not limited to, humans, mice, rats, guinea pigs, monkeys, dogs, cats, horses, cows, pigs, sheep, and poultry. In one embodiment, the mammal is a human.

The term “package insert” is used to refer to instructions customarily included in commercial packages of therapeutic products that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound. Exemplary salts include, but are not limited, to bismesylate, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition is compatible chemically and/or toxicologically with the other ingredients comprising a formulation and/or the patient being treated therewith.

The term “synergistic” as used herein refers to a therapeutic combination which is more effective than the additive effects of the two or more single agents. A determination of a synergistic interaction may be based on the results obtained from the assays known in the art. The results of these assays can be analyzed using the Chou and Talalay combination method and Dose-Effect Analysis with CalcuSyn software in order to obtain a Combination Index (Chou and Talalay, 1984, Adv. Enzyme Regul. 22:27-55). The combinations provided herein can be analyzed utilizing a standard program for quantifying synergism, additivism, and antagonism among anticancer agents. An example program is that described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. Thus, in embodiments, the combined amount of the active ingredients are effective in providing a synergistic effect (also referred to herein as a synergistically effective amount). A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehár et al. 2007, Molecular Systems Biology 3:80). BLISS scores quantify degree of potentiation from single agents and a positive BLISS score (greater than 0) suggests greater than simple additivity. A cumulative positive BLISS score greater than 250 is considered strong synergy observed within the concentration ranges tested. An HSA score (greater than 0) suggests a combination effect greater than the maximum of the single agent responses at corresponding concentrations.

In addition to providing improved treatment for a given hyperproliferative disorder, administration of certain combinations of the invention may improve the quality of life for a patient compared to the quality of life experienced by the same patient receiving a different treatment. For example, administration of a combination to a patient may provide an improved quality of life compared to the quality of life the same patient would experience if they received only one of the individual agents as therapy. For example, the combined therapy with a combination described herein may lower the dose of therapeutic agents needed. The combination therapy may also decrease or eliminate the need for the use of chemotherapeutic agents and the side-effects associated with high-dose chemotherapeutic agents (e.g. nausea, vomiting, hair loss, rash, decreased appetite, weight loss, etc.). The combination may also cause reduced tumor burden and the associated adverse events, such as pain, organ dysfunction, weight loss, etc. Accordingly, one aspect of the invention provides a combination for therapeutic use for improving the quality of life of a patient treated for a hyperproliferative disorder with an agent described herein.

One aspect includes a method of tumor growth inhibition (TGI) in a patient suffering from a cancer, comprising administering a combination described herein to the patient. In certain embodiments, the combination provides a synergistic effect.

In certain embodiments, the TGI of the combination is greater than the TGI of any one of GDC-0973 and GDC-0623 or MEHD7945A alone. In certain embodiments, the TGI of the combination is about 10, 15, 20, 25, 30, 35, 40, 45, 50, 55, 60, 65, 70 or 75 percent greater than the TGI of the agents alone.

Methods of measuring TGI are known in the art. In one example method, average tumor volumes are determined and compared from the patient before and after treatment. Tumor volumes can be measured in two dimensions (length and width) using any method in the art, for example UltraCal IV calipers (Fred V. Fowler Company) or by PET (positron emission tomography), or by some other method. The formula tumor volume (mm³)=(length×width)×0.5 can be used. Measuring tumor volumes over multiple time periods can be done using a mixed-modeling Linear Mixed Effects (LME) approach (Pinheiro et al. 2009). This approach can address both repeated measurements (and multiple patients). Cubic regression splines can be used to fit a non-linear profile to the time courses of tumor volume at each dose level. These non-linear profiles can then be related to dose within the mixed model. Tumor growth inhibition as a percent of vehicle can be calculated as a percent area under the fitted curve (AUC) per day in relation to the vehicle, using the following formula:

$$\% TGI = 100 [ 1 - ( \frac{{AUC}_{treatment} / day}{{AUC}_{vehicle} / day} ) ]$$

Using this formula, a TGI value of 100% indicates tumor stasis, greater than about 1% but less than about 100% indicates tumor growth inhibition, and greater than about 100% indicates tumor regression.

### II. MEK and HER3/EGFR Inhibitors

A. MEK Inhibitors

The present invention relates to MEK inhibitors and their use in a combination therapy with HER3 and EGFR inhibitors. MEK inhibitors have been extensively reviewed (S. Price, Putative Allosteric MEK1 and MEK 2 inhibitors, Expert Opin. Ther. Patents, 2008 18(6):603; J. I. Trujillo, MEK Inhibitors: a patent review 2008-2010 Expert Opin. Ther. Patents 2011 21(7):1045. Preferably the MEK inhibitor is selected from GDC-0973 (cobimetinib), GDC-0623, AZD6244 (selumetinib), AZD8330, BAY 86-9766 (refametinib), GSK-1120212 (trametinib), ARRY-162, MSC1936369, MK162, TAK733 and PD-325901. Most preferably the MEK inhibitor is GDC-0973 (cobimetinib) or GDC-0623.

GDC-0973 is an orally available, potent and highly selective inhibitor of MEK1 and MEK2, central components of the RAS/RAF pathway. GDC-0973 has the Chemical Abstract Registration Number (CAS) 934660-93-2 and the chemical structure:

[chemical structure]

GDC-0623 has the Chemical Abstract Registration Number (CAS) 1168091-68-6 and the chemical structure:

[chemical structure]

A. Preparation of MEK Inhibitors: GDC-0973 and GDC-0623

The MEK inhibitor GDC-0973 (Formula I), or a pharmaceutically acceptable salt thereof, can be prepared as described in in Example 22 of WO2007044515 or, alternatively, as described as described by Rice, et al. (K. D. Rice et al., Novel Carboxamide-Based Allosteric MEK inhibitors: Discovery and Optimization Efforts toward XL518 (GDC-0973, Med. Chem. Lett. 2012 3:416).

The MEK inhibitor GDC-0623 (Formula II), or a pharmaceutically acceptable salt thereof can be prepared, e.g., as described in Example 5 of WO2009/085983.

B. HER3/EGFR Inhibitors

The present invention relates to compounds which inhibit HER3, EGFR, or both HER3 and EGFR and their use in a combination therapy with a MEK inhibitor. The HER3, EGFR, and dual HER3/EGFR inhibitors can be an antibody or other antigen-binding protein, a small molecule, a nucleic acid (such as an siRNA), or any other such molecule.

In one embodiment, the combination therapy relates to HER3 inhibitors. Exemplary anti-HER3 antibodies are described in WO2011076683 (Mab205.10.1, Mab205.10.2, Mab205.10.3), U.S. Pat. No. 7,846,440; U.S. Pat. No. 7,705,130 and U.S. Pat. No. 5,968,511.

In one embodiment, the combination therapy relates to EGFR inhibitors. Examples of EGFR inhibitors include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR inhibitors include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR inhibitors include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N-8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Sugen); and AG1571 (SU 5271; Sugen).

In one embodiment, the combination therapy relates to bispecific HER3/EGFR inhibitors. In one embodiment, the bispecific HER3/EGFR inhibitor is a bispecific antibody. In one embodiment, the bispecific HER3/EGFR inhibitor is a bispecific antibody which comprises an antigen binding domain that specifically binds to both HER3 and EGFR. In one embodiment, the bispecific HER3/EGFR inhibitor is a bispecific antibody which comprises two identical antigen binding domains, each of which specifically binds to both HER3 and EGFR. Such antibodies are described in WO2010108127, US20100255010 and Schaefer et al, Cancer Cell, 20: 472-486 (2011). One such particular bispecific HER3/EGFR inhibitor comprising an antigen binding domain that specifically binds to both HER3 and EGFR is DL11f, also known as MEHD7945A. MEHD7945A is capable of binding to Domain III of EGFR and Domain III of HER3. MEHD7945A is also able to bind to Fcγ receptors and has the potential to elicit antibody-dependent cell-mediated cytotoxicity (ADCC). MEHD7945A shows potent anti-tumor activity in various nonclinical models, including models that are unresponsive to anti-EGFR therapeutics.

The dual-action antibody MEHD7945A which comprises two identical antigen binding domains, each of which specifically binds to both HER3 and EGFR can be prepared as described in WO 2010/108127 (see DL11f, e.g., FIG. 33) and Schaefer et al., Cancer Cell, 20, 472-486 (2011). The amino acid sequence for the heavy chain variable domain of MEHD7945A is provided as SEQ ID NO: 1 and the amino acid sequence for the light chain variable domain of MEHD7945A is provided in SEQ ID NO: 2.

In one embodiment, the bispecific HER3/EGFR antibody comprises an antigen-binding domain that specifically binds to HER3 and EGFR where the antibody comprises a V_{H} comprising one, two, and/or three of the HVRs of the amino acid sequence of SEQ ID NO: 1. In one embodiment, the bispecific HER3/EGFR antibody comprises an antigen-binding domain that specifically binds to HER3 and EGFR where the antibody comprises a V_{H} comprising one, two, and/or three of the HVRs of the amino acid sequence of SEQ ID NO: 1 and a V_{L} comprising one, two, and/or three of the HVRs of the amino acid sequence of SEQ ID NO: 2. In one embodiment, the bispecific HER3/EGFR antibody comprises an antigen-binding domain that specifically binds to HER3 and EGFR where the antibody comprises a V_{H} comprising all three HVRs of the amino acid sequence of SEQ ID NO: 1 and a V_{L} comprising all three of the HVRs of the amino acid sequence of SEQ ID NO: 2. In some embodiments, the HVRs are extended HVRs. In one specific embodiment, HVR-H1 comprises the amino acid sequence LSGDWIH (SEQ ID NO: 3), HVR-H2 comprises the amino acid sequence VGEISAAGGYTD (SEQ ID NO: 4), HVR-H3 comprises the amino acid sequence ARESRVSFEAAMDY (SEQ ID NO: 5), HVR-L1 comprises the amino acid sequence NIATDVA (SEQ ID NO: 6), HVR-L2 comprises the amino acid sequence SASF (SEQ ID NO: 7), and HVR-L3 comprises the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} having at least 80%, 85%, 90%, 91%, 92%, 93%, 94%, 95%, 96%, 97%, 98%, or 99% sequence identity to the amino acid sequence of SEQ ID NO: 1. In one specific embodiment, the bispecific HER3/EGFR comprising a V_{H} having at least 80%, 85%, 90%, 91%, 92%, 93%, 94%, 95%, 96%, 97%, 98%, or 99% sequence identity to the amino acid sequence of SEQ ID NO: 1 comprises a HVR-H1 comprising the amino acid sequence LSGDWIH (SEQ ID NO: 3), HVR-H2 comprising the amino acid sequence VGEISAAGGYTD (SEQ ID NO: 4), and HVR-H3 comprising the amino acid sequence ARESRVSFEAAMDY (SEQ ID NO: 5).

In one embodiment, the bispecific HER3/EGFR antibody comprises an antigen-binding domain that specifically binds to HER3 and EGFR where the antibody comprises a V_{L} having at least 80%, 85%, 90%, 91%, 92%, 93%, 94%, 95%, 96%, 97%, 98%, or 99% sequence identity to the amino acid sequence of SEQ ID NO: 2. In one specific embodiment, the bispecific HER3/EGFR comprising a V_{L} having at least 80%, 85%, 90%, 91%, 92%, 93%, 94%, 95%, 96%, 97%, 98%, or 99% sequence identity to the amino acid sequence of SEQ ID NO: 2 comprises a HVR-L1 comprising the amino acid sequence NIATDVA (SEQ ID NO: 6), HVR-L2 comprising the amino acid sequence SASF (SEQ ID NO: 7), and HVR-L3 comprising the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} having at least 80%, 85%, 90%, 91%, 92%, 93%, 94%, 95%, 96%, 97%, 98%, or 99% sequence identity to the amino acid sequence of SEQ ID NO: 1 and a V_{L} having at least 80%, 85%, 90%, 91%, 92%, 93%, 94%, 95%, 96%, 97%, 98%, or 99% sequence identity to the amino acid sequence of SEQ ID NO: 2. In one embodiment, the bispecific HER3/EGFR antibody comprising a V_{H} having at least 80%, 85%, 90%, 91%, 92%, 93%, 94%, 95%, 96%, 97%, 98%, or 99% sequence identity to the amino acid sequence of SEQ ID NO: 1 and a V_{L} having at least 80%, 85%, 90%, 91%, 92%, 93%, 94%, 95%, 96%, 97%, 98%, or 99% sequence identity to the amino acid sequence of SEQ ID NO: 2 comprises a HVR-H1 comprising the amino acid sequence LSGDWIH (SEQ ID NO: 3), HVR-H2 comprising the amino acid sequence VGEISAAGGYTD (SEQ ID NO: 4), and HVR-H3 comprising the amino acid sequence ARESRVSFEAAMDY (SEQ ID NO: 5), a HVR-L1 comprising the amino acid sequence NIATDVA (SEQ ID NO: 6), HVR-L2 comprising the amino acid sequence SASF (SEQ ID NO: 7), and HVR-L3 comprising the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} comprising the amino acid sequence of SEQ ID NO: 1. In one embodiment, the bispecific HER3/EGFR antibody comprises an antigen-binding domain that specifically binds to HER3 and EGFR where the antibody comprises a V_{L} comprising the amino acid sequence of SEQ ID NO: 2. In one embodiment, the bispecific HER3/EGFR antibody comprises an antigen-binding domain that specifically binds HER3 and EGFR where the antibody comprises a V_{H} comprising the amino acid sequence of SEQ ID NO: 1 and a V_{L} comprising the amino acid sequence of SEQ ID NO: 2.

In one embodiment, the bispecific HER3/EGFR antibody comprises an antigen-binding domain that specifically binds to HER3 and EGFR where the antibody comprises a heavy chain comprising the amino acid sequence of SEQ ID NO: 9. In one embodiment, the bispecific HER3/EGFR antibody comprises an antigen-binding domain that specifically binds to HER3 and EGFR where the antibody comprises a light chain comprising the amino acid sequence of SEQ ID NO: 10. In one embodiment, the bispecific HER3/EGFR antibody comprises an antigen-binding domain that specifically binds HER3 and EGFR where the antibody comprises a heavy chain comprising the amino acid sequence of SEQ ID NO: 9 and a light chain comprising the amino acid sequence of SEQ ID NO: 10.

In some embodiments, the bispecific HER3/EGFR antibody comprising an antigen-binding domain that specifically binds to EGFR and HER3 is a full length IgG1 antibody.

C. Antibody Preparation

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthiin, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall' Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol, 147: 86 (1991).) Human antibodies generated via human B-cell technology are also described in Li et al., Proc. Natl. Acad. Sci. USA, 103:3557-:1562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a traditional bispecific antibody comprising two antigen binding domains each specific for a distinct target. Multispecific antibodies are monoclonal antibodies that have binding specificities for at least two different sites. In certain embodiments, one of the binding specificities is for HER3 and the other is for any other antigen. In certain embodiments, bispecific antibodies may bind to two different epitopes of HER3. Bispecific antibodies may also be used to localize cytotoxic agents to cells which express HER3. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to HER3 as well as another, different antigen (see, US 2008/0069820, for example). Examples of such a bispecific HER3/EGFR inhibitor are described herein and include the exemplary DL11f (MEHD7945A) antibody.

7. Antibody Variants

In certain embodiments, amino acid sequence variants of the antibodies provided herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e g, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

1.) Recombinant Methods and Compositions

Antibodies may be produced using recombinant methods and compositions, e.g., as described in U.S. Pat. No. 4,816,567. In one embodiment, isolated nucleic acid encoding an anti-HER3/anti-EGFR antibody (including bispecific antibodies) described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody (including bispecific antibodies) is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody (including bispecific antibodies), nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### III. Compositions

Pharmaceutical compositions or formulations of the present invention include combinations as described herein.

The compounds described herein or a pharmaceutically acceptable salt thereof may exist in unsolvated as well as solvated forms with pharmaceutically acceptable solvents such as water, ethanol, and the like, and it is intended that the invention embrace both solvated and unsolvated forms.

The compound or a pharmaceutically acceptable salt thereof may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Pharmaceutical compositions encompass both the bulk composition and individual dosage units comprised of more than one (e.g., two) pharmaceutically active agents, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the herein-described method of treating a patient by administering a pharmaceutical composition of the present invention is also intended to encompass the administration of the bulk composition and individual dosage units.

Pharmaceutical compositions also embrace isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy.

The pharmaceutically acceptable salts of the compounds are formulated in accordance with standard pharmaceutical practice for use in a therapeutic combination for therapeutic treatment of hyperproliferative disorders (such as cancer, such as triple negative breast cancer) in mammals including humans (such as human males or females). The invention provides a pharmaceutical composition comprising a combination as described herein in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for administration may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds may be prepared for various routes and types of administration. For example, the compound or a pharmaceutically acceptable salt thereof having the desired degree of purity may optionally be mixed with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations will be dosed and administered in a fashion, e.g., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa.

Sustained-release preparations may be prepared. Suitable examples of sustained-release preparations include semipermeable matrices of solid hydrophobic polymers containing a compound or a pharmaceutically acceptable salt thereof, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D (−) 3-hydroxybutyric acid.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of combinations suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount GDC-0973 and GDC-0623, or a pharmaceutically acceptable salt thereof; and MEHD7945A. The amount of GDC-0973 and GDC-0623, or a pharmaceutically acceptable salt thereof; and MEHD7945A may be formulated in a pill, capsule, solution or suspension as a combined formulation. Alternatively, the combination may be formulated separately in a pill, capsule, solution or suspension for administration by alternation.

Formulations may be prepared according to any method known to the art for the manufacture of pharmaceutical compositions and such compositions may contain one or more agents including sweetening agents, flavoring agents, coloring agents and preserving agents, in order to provide a palatable preparation. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom. Tablet excipients of a pharmaceutical formulation may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tableting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner, including a mixture of at least one emulsifier with a fat or an oil, or with both a fat and an oil. Preferably, a hydrophilic emulsifier is included together with a lipophilic emulsifier which acts as a stabilizer. Together, the emulsifier(s) with or without stabilizer(s) make up an emulsifying wax, and the wax together with the oil and fat comprise an emulsifying ointment base which forms the oily dispersed phase of cream formulations. Emulsifiers and emulsion stabilizers suitable for use in the formulation include Tween® 60, Span® 80, cetostearyl alcohol, benzyl alcohol, myristyl alcohol, glyceryl mono-stearate and sodium lauryl sulfate.

Aqueous suspensions of the pharmaceutical formulations contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount(s) of active ingredient(s) that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising a combination described herein together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### IV. Combination Therapy

One aspect of the invention provides for a combination therapy for treatment of cancer in a patient wherein the combination therapy comprises administration of a MEK inhibitor, an EGFR inhibitor, and a HER3 inhibitor to the patient.

In one embodiment, the MEK inhibitor of the combination therapy is either GDC-0973 or GDC-0623. GDC-0973 and GDC-0623 are potent and highly selective small molecule allosteric inhibitors of MEK 1/2, the kinases that activate ERK 1/2 Inhibition of MEK 1/2 is a promising strategy to control the growth of tumors that are dependent on aberrant signaling in the MEK/ERK pathway. Preclinical studies have demonstrated that both inhibitors are effective in inhibiting the growth of tumor cells bearing activating B-RAF mutations that are associated with many tumor types, with GDC-0973 showing more activity in this model. FIG. 5. Preclinical studies have demonstrated that both inhibitors are effective in inhibiting the growth of tumor cells bearing activating Ras mutations that are associated with many tumor types, with GDC-0623 showing more activity in this model. FIG. 6.

In one embodiment, the HER3 inhibitor and the EGFR inhibitor functions are present in the same molecule, for example, a bispecific antibody capable of binding to and inhibiting the biological activity of both HER3 and EGFR. In one embodiment, the HER3 and EGFR inhibitor is a bispecific antibody which specifically binds to both HER3 and EGFR. In one embodiment, the HER3 and EGFR inhibitor is a bispecific antibody which comprises two identical antigen binding domains, each of which specifically binds to both HER3 and EGFR.

In one embodiment, the HER3 and EGFR bispecific antibody which comprises two identical antigen binding domains, each of which specifically binds to both HER3 and EGFR is antibody MEHD7945A. MEHD7945A blocks ligand binding to its EGFR and HER3 targets. The MEHD7945A antibody binds to EGFR with a Kd of about 1.9 nM and binds to HER3 with a Kd of about 0.4 mM. (See WO 2010/108127 and Schaefer, et al. Cancer Cell, 20: 472-486 (2011)). MEHD7945A inhibits EGFR and HER2/HER3-dependent signaling. Furthermore MEHD7945A, as a single agent, inhibits MAPK and PI3K signaling.

The combination of a MEK inhibitor with a HER3 and EGFR inhibitor or inhibitors provides a method of inhibiting both RAS/MEK and PI3K/AKT pathways and thus provides a more effective anti-cancer therapy. The combination therapy would also serve to prevent or delay the inherent or acquired resistance attributable to activation of the PI3K/AKT pathway observed with MEK inhibition and to prevent or delay inherent or acquired resistance mediated via RAS pathway activation. Furthermore, the combination therapy would serve to block two established EGFR-resistance mechanisms—KRAS mutations and HER3 activation.

The MEK inhibitor, HER3 inhibitor and EGFR inhibitor may be formulated in a single pharmaceutical composition. Alternatively, the combination may be present as two pharmaceutical compositions wherein a first pharmaceutical composition includes one of a MEK inhibitor, a HER3 inhibitor and an EGFR inhibitor and a second pharmaceutical composition comprising two of the MEK inhibitor, the HER3 inhibitor or the EGFR inhibitor, wherein the MEK inhibitor, the HER3 inhibitor and the EGFR inhibitor are not present in both the first pharmaceutical composition and the second pharmaceutical composition. In embodiments, the combination may be present as two pharmaceutical compositions wherein a first pharmaceutical composition includes a MEK inhibitor and a second pharmaceutical composition comprises a HER3 inhibitor and an EGFR inhibitor. In embodiments, the combination may be present as three pharmaceutical compositions, wherein each of the three pharmaceutical compositions include one of a MEK inhibitor, a HER3 inhibitor or a EGFR inhibitor.

When the combination comprises a dual HER3/EGFR inhibitor, such as MEHD7945A, the MEK inhibitor and the dual HER3/EGFR inhibitor may be formulated in a single pharmaceutical composition or the MEK inhibitor may be formulated in a first pharmaceutical composition and the dual HER3/EGFR inhibitor may be formulated in a second pharmaceutical composition.

As demonstrated in the Examples, the combination of MEHD7945A and cobimetinib results in robust activity in vitro and in vivo. In vitro signaling studies in colorectal cell lines demonstrate that the effect of the combination of MEHD7945A and cobimetinib on the inhibition of AKT and ERK signaling is superior compared to single-agent activity Inhibition of proliferation was also enhanced in the combination group. Increased in vivo efficacy was demonstrated in the combination group when compared to the single-agent groups in KRAS-mutant xenograft models of colon cancer, supporting the hypothesis that combined inhibition of the signaling receptors EGFR and HER3 and concurrent inhibition of the RAS/RAF/MEK pathway is necessary to prevent compensatory pathway activation and thereby enhance potency. Increased in vivo efficacy was seen in the combination group when compared to single-agent treatment in a pancreatic wild-type KRAS xenograft model, suggesting that the combination of MEHD7945A and cobimetinib is also beneficial in pancreatic cancers.

The combination may be employed in combination with chemotherapeutic agents for the treatment of a hyperproliferative disease or disorder, including tumors, cancers, and neoplastic tissue, along with pre-malignant and non-neoplastic or non-malignant hyperproliferative disorders. In certain embodiments, a combination is combined in a dosing regimen as combination therapy, with another compound that has anti-hyperproliferative properties or that is useful for treating the hyperproliferative disorder. The additional compound of the dosing regimen preferably has complementary activities to the combination, and such that they do not adversely affect each other. Such compounds may be administered in amounts that are effective for the purpose intended. In one embodiment, the therapeutic combination is administered by a dosing regimen wherein the therapeutically effective amount of a MEK inhibitor compound (such as GDC-0973 or GDC-0623), or a pharmaceutically acceptable salt thereof is administered in a range from twice daily to once every three weeks (q3wk), and the therapeutically effective amount of HER3/EGFR inhibitor or inhibitors (such as MEHD7945A) is administered in a range from twice daily to once every three weeks.

The combination therapy may be administered as a simultaneous or sequential regimen. When administered sequentially, the combination may be administered in two or more administrations. The combined administration includes coadministration, using separate formulation, and consecutive administration in either order, wherein preferably there is a time period while both (or all) active agents simultaneously exert their biological activities.

In one specific aspect of the invention, the MEK inhibitor compound (such as GDC-0973 or GDC-0623), or a pharmaceutically acceptable salt thereof can be administered for a time period of about 1 to about 10 days after administration of the HER3/EGFR inhibitor or inhibitors (such as MEHD7945A) begins. In another specific aspect of the invention, the MEK inhibitor compound (such as GDC-0973 or GDC-0623), or a pharmaceutically acceptable salt thereof can be administered for a time period of about 1 to 10 days before administration of the HER3/EGFR inhibitor or inhibitors (such as MEHD7945A) begins. In another specific aspect of the invention, administration of the compound of the MEK inhibitor compound (such as GDC-0973 or GDC-0623), or a pharmaceutically acceptable salt thereof and administration of the HER3/EGFR inhibitor or inhibitors (such as MEHD7945A) begin on the same day.

In one specific aspect of the invention the HER3/EGFR inhibitor or inhibitors (such as MEHD7945A) can be administered for a time period of about 1 to about 10 days after administration of the MEK inhibitor compound (such as GDC-0973 or GDC-0623), or a pharmaceutically acceptable salt thereof begins. In another specific aspect of the invention, the HER3/EGFR inhibitor or inhibitors (such as MEHD7945A) can be administered for a time period of about 1 to 10 days before administration of the MEK inhibitor compound (such as GDC-0973 or GDC-0623), or a pharmaceutically acceptable salt thereof begins. In another specific aspect of the invention, administration of the HER3/EGFR inhibitor or inhibitors (such as MEHD7945A) and administration of the MEK inhibitor compound (such as GDC-0973 or GDC-0623), or a pharmaceutically acceptable salt thereof begin on the same day.

Suitable dosages for any of the above coadministered agents are those presently used and may be lowered due to the combined action (synergy) of the newly identified agent and other chemotherapeutic agents or treatments, such as to increase the therapeutic index or mitigate toxicity or other side-effects or consequences.

In a particular embodiment of anti-cancer therapy, the therapeutic combination may combined with surgical therapy and radiotherapy. The amounts of the combination and the relative timings of administration will be selected in order to achieve the desired combined therapeutic effect.

### V. Dosage Regimes for the Combination Therapy

A dose of MEK inhibitor compound of formula I or II, or a pharmaceutically acceptable salt thereof, to treat human patients may range from about 20 mg to about 1600 mg of the compound. A typical dose may be about 50 mg to about 800 mg of the compound. A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy.

A dose to treat human patients with an antibody, such as MEHD7945A, may range from about 0.05 mg/kg to about 30 mg/kg. Thus, one or more doses of about 0.5 mg/kg, 2.0 mg/kg, 4.0 mg/kg, 10 mg/kg, 12 mg/kg, 13 mg/kg, 14 mg·kg, 15 mg/kg, 20 mg/kg, 25 mg/kg, or 30 mg/kg (or any combination thereof) may be administered to the patient. Such doses may be administered daily or intermittently, e.g. every week, every two weeks, or every three weeks.

In one particular embodiment, the dose to a human patient is 1100 mg of MEHD7945A administered by IV every two weeks (Q2W) and 40 mg orally daily (QD) of GDC-0973 (cobimetinib). In another particular embodiment, the dose to a human patient is 1100 mg of MEHD7945A IV Q2W and 50 mg orally QD of GDC-0973 (cobimetinib). In another particular embodiment, the dose to a human patient is 1100 mg of MEHD7945A IV Q2W and 60 mg orally QD of GDC-0973 (cobimetinib). In another particular embodiment, the dose to a human patient is 1100 mg of MEHD7945A IV Q2W and 70 mg orally QD of GDC-0973 (cobimetinib). In another particular embodiment, the dose to a human patient is 1100 mg of MEHD7945A IV Q2W and 80 mg orally QD of GDC-0973 (cobimetinib). In these particular embodiments, patients receive 1100 mg of MEHD7945A IV Q2W; GDC-0973 (cobimetinib) will be administered for 21 consecutive days followed by 7 days off.

In another particular embodiment, the dose to a human patient is 1100 mg of MEHD7945A administered by IV every two weeks (Q2W) and 40 mg of GDC-0973 (cobimetinib) administered orally once a week. In another particular embodiment, the dose to a human patient is 1100 mg of MEHD7945A IV Q2W and 50 mg orally QD of GDC-0973 (cobimetinib) administered orally once a week. In another particular embodiment, the dose to a human patient is 1100 mg of MEHD7945A IV Q2W and 60 mg orally QD of GDC-0973 (cobimetinib) administered orally once a week. In another particular embodiment, the dose to a human patient is 1100 mg of MEHD7945A IV Q2W and 70 mg orally QD of GDC-0973 (cobimetinib) administered orally once a week. In another particular embodiment, the dose to a human patient is 1100 mg of MEHD7945A IV Q2W and 80 mg orally QD of GDC-0973 (cobimetinib) administered orally once a week.

In another particular embodiment, the dose to a human patient is 1100 mg of MEHD7945A administered by IV every two weeks (Q2W) and 40 mg of GDC-0973 (cobimetinib) administered orally twice a week. In another particular embodiment, the dose to a human patient is 1100 mg of MEHD7945A IV Q2W and 50 mg orally QD of GDC-0973 (cobimetinib) administered orally twice a week. In another particular embodiment, the dose to a human patient is 1100 mg of MEHD7945A IV Q2W and 60 mg orally QD of GDC-0973 (cobimetinib) administered orally twice a week. In another particular embodiment, the dose to a human patient is 1100 mg of MEHD7945A IV Q2W and 70 mg orally QD of GDC-0973 (cobimetinib) administered orally twice a week. In another particular embodiment, the dose to a human patient is 1100 mg of MEHD7945A IV Q2W and 80 mg orally QD of GDC-0973 (cobimetinib) administered orally twice a week.

In another particular embodiment, the dose to a human patient is 1100 mg of MEHD7945A administered by IV every two weeks (Q2W) and 40 mg of GDC-0973 (cobimetinib) administered orally three times a week. In another particular embodiment, the dose to a human patient is 1100 mg of MEHD7945A IV Q2W and 50 mg orally QD of GDC-0973 (cobimetinib) administered orally three times a week. In another particular embodiment, the dose to a human patient is 1100 mg of MEHD7945A IV Q2W and 60 mg orally QD of GDC-0973 (cobimetinib) administered orally three times a week. In another particular embodiment, the dose to a human patient is 1100 mg of MEHD7945A IV Q2W and 70 mg orally QD of GDC-0973 (cobimetinib) administered orally three times a week. In another particular embodiment, the dose to a human patient is 1100 mg of MEHD7945A IV Q2W and 80 mg orally QD of GDC-0973 (cobimetinib) administered orally three times a week.

In another particular embodiment, the dose to a human patient is 1100 mg of MEHD7945A administered by IV every two weeks (Q2W) and 40 mg of GDC-0973 (cobimetinib) administered orally four times a week. In another particular embodiment, the dose to a human patient is 1100 mg of MEHD7945A IV Q2W and 50 mg orally QD of GDC-0973 (cobimetinib) administered orally four times a week. In another particular embodiment, the dose to a human patient is 1100 mg of MEHD7945A IV Q2W and 60 mg orally QD of GDC-0973 (cobimetinib) administered orally four times a week. In another particular embodiment, the dose to a human patient is 1100 mg of MEHD7945A IV Q2W and 70 mg orally QD of GDC-0973 (cobimetinib) administered orally four times a week. In another particular embodiment, the dose to a human patient is 1100 mg of MEHD7945A IV Q2W and 80 mg orally QD of GDC-0973 (cobimetinib) administered orally four times a week.

In another particular embodiment, the dose to a human patient is 1100 mg of MEHD7945A administered by IV every two weeks (Q2W) and 40 mg of GDC-0973 (cobimetinib) administered orally five times a week. In another particular embodiment, the dose to a human patient is 1100 mg of MEHD7945A IV Q2W and 50 mg orally QD of GDC-0973 (cobimetinib) administered orally five times a week. In another particular embodiment, the dose to a human patient is 1100 mg of MEHD7945A IV Q2W and 60 mg orally QD of GDC-0973 (cobimetinib) administered orally five times a week. In another particular embodiment, the dose to a human patient is 1100 mg of MEHD7945A IV Q2W and 70 mg orally QD of GDC-0973 (cobimetinib) administered orally five times a week. In another particular embodiment, the dose to a human patient is 1100 mg of MEHD7945A IV Q2W and 80 mg orally QD of GDC-0973 (cobimetinib) administered orally five times a week.

In another particular embodiment, the dose to a human patient is 1100 mg of MEHD7945A administered by IV every two weeks (Q2W) and 40 mg of GDC-0973 (cobimetinib) administered orally six times a week. In another particular embodiment, the dose to a human patient is 1100 mg of MEHD7945A IV Q2W and 50 mg orally QD of GDC-0973 (cobimetinib) administered orally six times a week. In another particular embodiment, the dose to a human patient is 1100 mg of MEHD7945A IV Q2W and 60 mg orally QD of GDC-0973 (cobimetinib) administered orally six times a week. In another particular embodiment, the dose to a human patient is 1100 mg of MEHD7945A IV Q2W and 70 mg orally QD of GDC-0973 (cobimetinib) administered orally six times a week. In another particular embodiment, the dose to a human patient is 1100 mg of MEHD7945A IV Q2W and 80 mg orally QD of GDC-0973 (cobimetinib) administered orally six times a week.

In another particular embodiment, the dose to a human patient is 400 mg of MEHD7945A administered by IV once a week (QW) and 40 mg of GDC-0973 (cobimetinib) administered orally once a week. In another particular embodiment, the dose to a human patient is 400 mg of MEHD7945A administered by IV QW and 50 mg orally QD of GDC-0973 (cobimetinib) administered orally once a week. In another particular embodiment, the dose to a human patient is 400 mg of MEHD7945A administered by IV QW and 60 mg orally QD of GDC-0973 (cobimetinib) administered orally once a week. In another particular embodiment, the dose to a human patient is 400 mg of MEHD7945A administered by IV QW and 70 mg orally QD of GDC-0973 (cobimetinib) administered orally once a week. In another particular embodiment, the dose to a human patient is 400 mg of MEHD7945A administered by IV QW and 80 mg orally QD of GDC-0973 (cobimetinib) administered orally once a week.

In another particular embodiment, the dose to a human patient is 400 mg of MEHD7945A administered by IV QW and 40 mg of GDC-0973 (cobimetinib) administered orally twice a week. In another particular embodiment, the dose to a human patient is 400 mg of MEHD7945A administered by IV QW and 50 mg orally QD of GDC-0973 (cobimetinib) administered orally twice a week. In another particular embodiment, the dose to a human patient is 400 mg of MEHD7945A administered by IV QW and 60 mg orally QD of GDC-0973 (cobimetinib) administered orally twice a week. In another particular embodiment, the dose to a human patient is 400 mg of MEHD7945A administered by IV QW and 70 mg orally QD of GDC-0973 (cobimetinib) administered orally twice a week. In another particular embodiment, the dose to a human patient is 400 mg of MEHD7945A administered by IV QW and 80 mg orally QD of GDC-0973 (cobimetinib) administered orally twice a week.

In another particular embodiment, the dose to a human patient is 400 mg of MEHD7945A administered by IV QW and 40 mg of GDC-0973 (cobimetinib) administered orally three times a week. In another particular embodiment, the dose to a human patient is 400 mg of MEHD7945A administered by IV QW and 50 mg orally QD of GDC-0973 (cobimetinib) administered orally three times a week. In another particular embodiment, the dose to a human patient is 400 mg of MEHD7945A administered by IV QW and 60 mg orally QD of GDC-0973 (cobimetinib) administered orally three times a week. In another particular embodiment, the dose to a human patient is 400 mg of MEHD7945A administered by IV QW and 70 mg orally QD of GDC-0973 (cobimetinib) administered orally three times a week. In another particular embodiment, the dose to a human patient is 400 mg of MEHD7945A administered by IV QW and 80 mg orally QD of GDC-0973 (cobimetinib) administered orally three times a week.

In another particular embodiment, the dose to a human patient is 400 mg of MEHD7945A administered by IV QW and 40 mg of GDC-0973 (cobimetinib) administered orally four times a week. In another particular embodiment, the dose to a human patient is 400 mg of MEHD7945A administered by IV QW and 50 mg orally QD of GDC-0973 (cobimetinib) administered orally four times a week. In another particular embodiment, the dose to a human patient is 400 mg of MEHD7945A administered by IV QW and 60 mg orally QD of GDC-0973 (cobimetinib) administered orally four times a week. In another particular embodiment, the dose to a human patient is 400 mg of MEHD7945A administered by IV QW and 70 mg orally QD of GDC-0973 (cobimetinib) administered orally four times a week. In another particular embodiment, the dose to a human patient is 400 mg of MEHD7945A administered by IV QW and 80 mg orally QD of GDC-0973 (cobimetinib) administered orally four times a week.

In another particular embodiment, the dose to a human patient is 400 mg of MEHD7945A administered by IV QW and 40 mg of GDC-0973 (cobimetinib) administered orally five times a week. In another particular embodiment, the dose to a human patient 400 mg of MEHD7945A administered by IV QW and 50 mg orally QD of GDC-0973 (cobimetinib) administered orally five times a week. In another particular embodiment, the dose to a human patient is 400 mg of MEHD7945A administered by IV QW and 60 mg orally QD of GDC-0973 (cobimetinib) administered orally five times a week. In another particular embodiment, the dose to a human patient is 400 mg of MEHD7945A administered by IV QW and 70 mg orally QD of GDC-0973 (cobimetinib) administered orally five times a week. In another particular embodiment, the dose to a human patient is 400 mg of MEHD7945A administered by IV QW and 80 mg orally QD of GDC-0973 (cobimetinib) administered orally five times a week.

In another particular embodiment, the dose to a human patient is 400 mg of MEHD7945A administered by IV QW and 40 mg of GDC-0973 (cobimetinib) administered orally six times a week. In another particular embodiment, the dose to a human patient is 400 mg of MEHD7945A administered by IV QW and 50 mg orally QD of GDC-0973 (cobimetinib) administered orally six times a week. In another particular embodiment, the dose to a human patient is 400 mg of MEHD7945A administered by IV QW and 60 mg orally QD of GDC-0973 (cobimetinib) administered orally six times a week. In another particular embodiment, the dose to a human patient is 400 mg of MEHD7945A administered by IV QW and 70 mg orally QD of GDC-0973 (cobimetinib) administered orally six times a week. In another particular embodiment, the dose to a human patient is 400 mg of MEHD7945A administered by IV QW and 80 mg orally QD of GDC-0973 (cobimetinib) administered orally six times a week.

### VI. Methods of Treatment

Therapeutic combinations provided herein are useful for treating diseases, conditions and/or disorders including, but not limited to, those modulated by AKT kinase in a patient. Cancers that can be treated according to the methods of this invention include, but are not limited to, colorectal, mesothelioma, endometrial, pancreatic, breast, lung, ovarian, prostate, melanoma, gastric, colon, renal, head and neck, and glioblastoma.

Combinations of the invention may provide improved effects against certain cancer phenotypes. For example, certain combinations of the invention may provide improved effects against cancers associated with RAS mutation (such as KRAS mutations), EGFR mutations (such as T790M), PTEN mutation (or low or null status), AKT mutation (or high pAKT expression or amplification levels), PI3K mutation, or a combination of the above. In one embodiment, the cancer comprises a KRAS mutation at position 12 or 13. In certain embodiments, the KRAS mutation is G12A, G12C, G12D, G12R, G12S, G12V, G13C, or G13D.

Accordingly, certain combinations described herein may be particularly useful against these types of cancers. GDC-0973 has been shown to have improved efficacy against KRAS driven tumors which are common in colon, pancreatic and lung tumors.

| | Proliferation EC₅₀ (μM) | | |
|---|---|---|---|
| | A375 | HCT115 | KRAS^{G13D}/BRAF^{V600E} |
| Compound | (BRAF^{V600E}) | (KRAS^{G13D}) | EC₅₀ |
| GDC-0973 | 0.005 | 0.520 | 104 |
| GDC-0623 | 0.007 | 0.042 | 6 |

PTEN null (or low) status may be measured by any suitable means as is known in the art. In one example, 1HC is used. Alternatively, Western blot analysis can be used. Antibodies to PTEN are commercially available (Cell Signaling Technology, Beverly, Mass., Cascade Biosciences, Winchester, Mass.). Example procedures for IHC and Western blot analysis for PTEN status are described in Neshat, M. S. et al Enhanced sensitivity of PTEN-deficient tumors to inhibition of FRAP/mTOR, Proc. Natl. Acad. Sci. USA 98, 10314-10319 (2001) and Perren, A., et. al Immunohistochemical Evidence of Loss of PTEN Expression in Primary Ductal Adenocarcinomas of the Breast, American Journal of Pathology, Vol. 155, No. 4, October 1999. Additionally, cancers associated with AKT mutation or with PI3K mutation can be identified using techniques that are known in the art.

The level of activation or phosphorylation of AKT (“pAKT”) compared to the level of non-activated or non-phosphorylated AKT in a given sample can be measured by methods known in the art. The pAKT status can be expressed in terms of a ratio (e.g. amount of pAKT in a tumor cell divided by amount pAKT in a non-tumorous cell of the same type) or a subtraction (e.g, amount of pAKT in a tumor cell minus amount pAKT in the cell or in a non-tumorous cell of the same type). The pAKT profile can also be expressed in terms of the level of activation of the pathway by measuring amounts of phosphorylated downstream targets of AKT (for example, pGSK PRAS40), A high pAKT refers to activation or phosphorylation levels of overall. AKT in the sample that are higher than a baseline value. In one example, the baseline value is the basal levels of pAKT for a given cell type. In another example, the baseline value is average or mean level of pAKT in a given population of sample cells, for example non-cancerous or cells. In another example, a high pAKT refers to a tumor cell. that over-expresses or -amplified phosphorylated or activated AKT in the cell, when compared to an average of normal, healthy (e.g. non-tumorous) cells of the same type from either the same mammal or a patient population. The pAKT profile can also be used in conjunction with other markers, for example FOXO3a localization profiles, for predicting efficacy of certain PI3k/AKT kinase pathway inhibitors. Kits for testing for the presence of PI3k, KRAS and AKT mutations are commercially available (Qiagen).

In one specific aspect, the invention provides a method for treating a patient having a cancer that is associated with PTEN mutation or loss of expression, AKT mutation or amplification, PI3K mutation or amplification, or a combination thereof comprising administering a combination of the invention to the patient. In another aspect, the invention provides a method for identifying a patient having a cancer that that can be treated with a combination of the invention comprising determining if the patient's cancer is associated with PTEN mutation or loss of expression, AKT mutation or amplification, PI3K mutation or amplification, or a combination thereof, wherein association of the patient's cancer with PTEN mutation or loss of expression, AKT mutation or amplification, PI3K mutation or amplification or amplification or a combination thereof is indicative of a cancer that can be treated with a combination of the invention. In a further aspect, the invention provides a method further comprising treating the patient so identified with a combination of the invention. In one embodiment, the cancer is ovarian, breast, melanoma, colon, head and neck, or non-small cell lung cancer.

### VII. Articles of Manufacture

In another embodiment of the invention, an article of manufacture, or “kit”, containing a combination useful for the treatment of the diseases and disorders described above is provided. In one embodiment, the kit comprises a container and a combination described herein.

The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a combination, or a formulation thereof, which is effective for treating the condition and may have a sterile access port (for example, the container may be an intravenous solution bag or a vial having a stopper pierceable by a hypodermic injection needle). The label or package insert indicates that the composition is used for treating the condition of choice, such as cancer. In one embodiment, the label or package inserts indicates that the composition comprising the combination can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the combination, and, if present, the second pharmaceutical formulation. For example, if the kit comprises a first composition comprising GDC-0973 or GDC-0623, or a pharmaceutically acceptable salt thereof and a second pharmaceutical formulation comprising MEHD7945A, the kit may further comprise directions for the simultaneous, sequential or separate administration of the first and second pharmaceutical compositions to a patient in need thereof.

In another embodiment, the kits are suitable for the delivery of solid oral forms of a combination, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with GDC-0973 or GDC-0623, or a pharmaceutically acceptable salt thereof contained therein; (b) a second container with MEHD7945A and (c) a third container with a third pharmaceutical formulation contained therein, wherein the third pharmaceutical formulation comprises another compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of GDC-0973 or GDC-0623, or a pharmaceutically acceptable salt thereof and MEHD7945A, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### VIII. Examples

In order to illustrate the invention, the following examples are included. However, it is to be understood that these examples do not limit the invention and are only meant to suggest a method of practicing the invention.

### Example 1

### MEHD7945A is Specific for Both HER3 and EGFR

MEHD7945A is an antibody comprising an antigen-binding domain that has binding specificity for both EGFR and HER3. WO 2010/108127 and Schaefer, et al. Cancer Cell, 20: 472-486 (2011). Typically, bitargeting agents are constructed by linking two distinct antigen-binding modules, each module being able to bind to only one antigen. In contrast, in MEHD7945A, each module (Fab) can bind either of two antigens, thus having the potential to elicit enhanced binding affinity from an avidity effect. To confirm that each of the two identical Fabs of MEHD7945A can bind either EGFR or HER3, a competitive binding assay was performed. MEHD7945A binding to immobilized HER3-ECD was reduced in a dose-dependent manner with increasing amounts of EGFR-ECD. Conversely, MEHD7945A was competed from immobilized EGFR-ECD by soluble HER3-ECD protein. As expected, given their relative binding constants, higher concentrations of soluble EGFR-ECD were needed to compete with binding of MEHD7945A to immobilized HER3-ECD (FIG. 1). The results in FIG. 1 are expressed as MEHD7945A concentration versus OD. The assays examined the binding of MEHD7945A to immobilized HER3-ECD or EGFR-ECD, as indicated, in the presence of indicated soluble competitor: 1×=0.02 μg/ml, 10×=0.2 μg/ml, 100×=2 μg/ml, 1000×=20 μg/ml. Results in FIG. 1 are expressed as DL11f concentration versus OD.

### Example 2

### MEHD7945A Inhibits EGFR and HER2/HER3—Dependent Signaling

The dual activity of MEHD7945A in cell signaling assays was determined To assess the inhibitory function on HER3, MCF-7 cells for which NRG treatment potently activates the HER2/HER3 pathway were used. Treatment with MEHD7945A prior to NRG stimulation potently inhibited the phosphorylation of HER3 in a dose-dependent manner, and markedly decreased the phosphorylation of AKT and ERK1/2 (FIG. 2A). MEHD7945A inhibited phosphorylation of HER3 with an IC50 of 0.05 μg/ml, phosphorylation of AKT with an IC50 value of 0.19 μg/ml, and phosphorylation of ERK1/2 with an IC50 value of 1.13 μg/ml. Treatment with a monospecific antibody against HER3, anti-HER3, that has comparable binding affinity to HER3 achieved similar results. Anti-HER3 inhibited phosphorylation of HER3 with an IC50 of 0.12 μg/ml, phosphorylation of AKT with an IC50 value of 0.74 μg/ml, and phosphorylation of ERK1/2 with an IC50 value of 1.83 μg/ml. EGFR-NR6 cells were pretreated with MEHD7945A prior to ligand stimulation and it was determined that DL11f inhibited phosphorylation of EGFR and ERK1/2 with IC50 values of 0.03 and 0.16 μg/ml, respectively (FIG. 2B). The monospecific EGFR antibody cetuximab was more effective in inhibiting phosphorylation of EGFR and downstream signaling molecules, which was likely due to the higher binding affinity to EGFR Moreover, betacellulin- and amphiregulin-induced EGFR phosphorylation was also inhibited by MEHD7945A. MEHD7945A inhibited ERK1/2 and AKT pathways as potently as the combination of anti-HER3 and cetuximab in A431 and BxPC3 cells.

The assays were performed as follows. MCF-7 cells treated with indicated concentrations of MEHD7945A or anti-HER3 were stimulated with 0.5 nM NRG for 10 min. Cell lysates were immunoblotted to detect pHER3 (Tyr1289), pAKT (Ser473), pERK1/2 (Thr202/Tyr204), and total HER3. FIG. 2A. EGFR-NR6 cells treated with indicated concentrations of MEHD7945A or cetuximab for 1 hr prior stimulation with 5 nM TGF-a for 10 min. Cell lysates were subjected to immunoblotting to detect, pERK1/2 (Thr202/Tyr204), total EGFR, and phosphorylated EGFR. Since EGFR-NR6 cells only express EGFR all potential phosphorylation sites of EGFR were detected using a pTyr antibody.

### Example 3

### MEHD7945A is Active in Numerous Cancer Models

### In Vivo Activity in Fadu Xenograft Model, a Head and Neck Squamous Cell Carcinoma Model

MEHD7945A, a commercially available anti-EGFR antibody, and an anti-HER3 antibody were tested in mice with established tumors derived from Fadu cells (ATCC HTB-43, Manassas, Va.) 5×10⁶ FaDu cells were inoculated subcutaneously in CB17 SCID mice. Animals with similarly sized tumors were randomized into treatment cohorts (n=9/group) as follows: Vehicle (MEHD7945A formulation buffer), anti-EGFR antibody (25 mg/kg), anti-HER3 antibody (50 mg/kg), and MEHD7945A (25 mg/kg). Treatments were administered intraperitoneally, beginning with a 2× loading dose (50 or 100 mg/kg respectively) on the day of randomization and continuing weekly for a total of four treatments. As shown in FIG. 3, MEHD7945A is active in the FaDu head and neck cancer model and is more effective in inhibiting tumor growth than either an anti-EGFR specific or an anti-HER3 specific antibody.

### MEHD7945A is Active in Additional Cancer Types

FIG. 4 provides a summary of the some of the additional cancer types in which MEHD7945A shows activity as well as the relative activity of cetuximab or a monospecific anti-HER3 antibody on the cancer types. Details of the assays used to generate this summary are provided in WO 2010/108127. In brief, mice were treated with 25 mg/kg MEHD7945A, 25 mg/kg cetuximab, 50 mg/kg anti-HER3 or the combination of 25 mg/kg cetuximab plus 50 mg/kg anti-HER3, once a week for 4 cycles. MAXF449, OVXF550 and LX983 were treated with 30 mg/kg MEHD7945A, 30 mg/kg cetuximab, 60 mg/kg anti-HER3 or the combination of 30 mg/kg cetuximab plus 60 mg/kg anti-HER3, once a week for 4 cycles. Initial dose was a 2× loading dose for all treatments. Percent of tumor growth inhibition (TGI) was calculated for each study based on the last day of study in which the majority of mice remained in the vehicle group. TGI below 25% is indicated as −, TGI between 25-50% is indicated as +, TGI between 51-75% is indicated as ++, and TGI of 76% and above as +++. NSCLC=non-small cell lung cancer, HNSSC=head and neck squamous cell carcinoma, CRC=colorectal cancer, n/a=non applicable. OVXF550, MAXF449 and LXF983 models are human patient derived transplant models.

### Example 4

### Combination of MEHD7945A with Either GDC-0973 or GDC-0623 Results in pERK Suppression that is Better than pERK Suppression Provided by Single Agent Therapy

Treatment with either of the MEK inhibitors as single agents resulted in an increase pAkt level while the combination therapy reduced the pAKT levels to baseline levels. Furthermore, the combination of MEHD7945A with either GDC-0973 or GDC-0623 results in pERK suppression in a Kras mutant model better than single agent therapy. FIG. 7. In this assay MEHD7945A was present in 10 mg/ml, GDC-0973 in 1 μM, GDC-0623 in μM, and heregulin (HRG) in 10 nM.

### Example 5

### Combined Therapy with MEHD7945A and Either GDC-0973 or GDC-0623 was Superior to Monotherapy in Preclinical Models of CRC KRAS Mutant Cancer

Mouse LS180 xenograft tumor models of KRAS mutant colorectal cancer were treated with MEHD7945A, GDC-0973 and GDC-0623 as single agents and in combinations consisting of MEHD7945A with GDC-0973 and MEHD7945A with GDC-0623. The treatment groups were as follows: 01—vehicle control; 03—GDC-0973 (10 mg/kg, PO, QD); 04—GDC-0623 (5 mg/kg, PO, QD); 06—MEHD7945A (25 mg/kg, IV, QW); 08—GDC-0973 (10 mg/kg, PO, QD)+MEHD7945A (25 mg/kg, IV, QW); 09—GDC-0623 (5 mg/kg, PO, QD)+MEHD7945A (25 mg/kg, IV, QW).

The tumor volume was measured over the course of treatment and the results are shown in FIG. 8. As shown in FIG. 8, the combination of MEHD7945A with either GDC-0973 or GDC-0623 was superior to single agent treatment.

### Example 6

### In Vitro Effects of a Combination of MEHD7945a and GDC-0973 in KRAS Mutant Colorectal Cell Lines

Inhibition of the RAS/RAF/MEK and the PI3K/AKT pathways were explored in vitro using MEHD7945A and cobimetinib or the combination of both agents in KRAS-mutant colorectal cell lines. Two KRAS mutant colorectal cell lines were selected to assess the potential upregulation of phosphorylated AKT (pAKT) by cobimetinib due to the inhibition of negative feedback loops. Upregulation of pAKT upon MEK inhibition has been described in several cell systems (Mirzoeva et al. 2009; Diep et al 2011; Turke et al. 2012). Moreover, we investigated if the addition of MEHD7945A to cobimetinib treatment could enhance pAKT and pERK1/2 inhibition. LS180 cells were pretreated with 10 μg/mL MEHD7945A, 0.05 μM cobimetinib, or the combination thereof for one hour before stimulation with 5 nM TGFα for twelve minutes. DLD-1 cells were pretreated with 10 μg/mL MEHD7945A, 0.025 μM cobimetinib, or the combination thereof for 1 hour before stimulation with 5 nM TGFα for 12 minutes. Cell lysates were immunoblotted to detect phosphorylation of EGFR (pEGFR1068), phosphorylation of AKT (pAKTS473), and phosphorylation of ERK1/2 (pERK1/2 T202/Y204), and total protein levels of EGFR, AKT, or ERK1/2. The results are shown in FIG. 9 (left panel=LS180 cells, right panel=DLD-1 cells) (EGFR=epidermal growth factor receptor; ERK=extracellular signal regulated kinase; p=phosphorylated; TGFα=transforming growth factor α)

TGFα-stimulated LS180 or DLD-1 cells that were treated with cobimetinib showed increased phosphorylation of AKT (FIG. 9, Lane 4 compared to control lysate (Lane 2) which suggests the presence of a MEK inhibitor-induced feedback loop (Mirzoeva et al. 2009; Diep et al 2011; Turke et al. 2012).

Only partial inhibition of ERK1/2 phosphorylation with low doses of cobimetinib (0.05 μM for LS180 cells and 0.025 μM for DLD-1 cells (see FIG. 9 left and right panels, respectively) was achieved. However, low doses of combined cobimetinib plus MEHD7945A resulted in strong downregulation of pERK and pAKT in both cell lines (see FIG. 9, Lane 5). In FIG. 9, the left panel displays LS180 cells pretreated with 10 μg/mL MEHD7945A, 0.05 μM cobimetinib, or the combination for 1 hour before stimulation with 5 nM TGFα for 12 minutes. Right panel displays DLD-1 cells pretreated with 10 λg/mL MEHD7945A, 0.025 μM cobimetinib, or the combination for 1 hour before stimulation with 5 nM TGFα for 12 minutes. Cell lysates were immunoblotted to detect phosphorylation of EGFR (pEGFR1068), phosphorylation of AKT (pAKTS473), and phosphorylation of ERK1/2 (pERK1/2 T202/Y204), and total protein levels of EGFR, AKT, or ERK1/2.

To test the anti-proliferative effect of combined inhibition of MEK1/2 and EGFR/HER3 in a KRAS-mutant cell line, LS180 cells were treated with increasing concentrations of cobimetinib (0.17-10,000 nM) in the presence or absence of 5 μg/mL of MEHD7945A. The combination of MEHD7945A and cobimetinib resulted in stronger reduction of cell viability when compared to the anti-proliferative effect of cobimetinib alone. The results are shown in FIG. 10 (results are expressed as RFU (relative fluorescence unit) plotted against SMI (small molecule inhibitor) concentration. For data analysis, a 4-parameter curve-fitting program was used. Data are representative of three independent experiments).

### Example 7

### Combination Studies of MEHD7945A with Cobimetinib in LS180 and DLD-1 Xenograft Models

A combination study of MEHD7945A and cobimetinib was conducted in the KRAS-mutant colorectal xenograft models LS180 and DLD-1. Both of these models were selected because of their KRAS-mutant status and their EGFR and HER3 expression. Cobimetinib was administered as an aqueous solution orally at 3 or 10 mg/mL once daily for 21 days. MEHD7945A was administered IV once a week until Day 21 was reached. Tumor sizes and body weights were recorded twice weekly over the course of the study. Mice were promptly euthanized when tumor volume exceeded 2000 mm3 or if body weight loss was 20% of their starting weight.

To appropriately analyze the repeated measurement of tumor volumes from the same animals over time, a mixed-modeling approach was used (Pinheiro et al. 2009). This approach addressed both repeated measurements and modest drop-out rates due to non-treatment-related termination of animals before study end. This analysis was used to determine tumor growth inhibition as a percentage of vehicle (% TGI) or time to tumor progression (TTP).

### LS180 Model

After randomization, LS180 tumor bearing mice were given oral (PO) gavage doses of 0 (vehicle), 3, or 10 mg/kg cobimetinib (expressed as free-base equivalents) once a day (QD) for 21 days. Mice were given 25 mg/kg of MEHD7945A via intravenous (IV) bolus injection once per week (QW) for a total of three injections. In groups that received both agents, cobimetinib was administered first and immediately followed by MEHD7945A.

Administration of cobimetinib at 3 or 10 mg/kg or MEHD7945A at 25 mg/kg resulted in 28%, 63%, and 44% TGI, respectively. Cobimetinib and MEHD7945A in combination had a stronger anti-tumor activity compared to single-agent activity. Cobimetinib at 3 and 10 mg/kg with MEHD7945A at 25 mg/kg resulted in 48% and 79% TGI, respectively. The data are shown in FIG. 11A and the study is summarized in FIG. 11B. In FIG. 11 CI=confidence interval; HB#8=histidine buffer 8; MCT=0.5% (w/v) methylcellulose, 0.2% (w/v) polysorbate 80; TGI=tumor growth inhibition; w/v=weight per volume.

### DLD-1

After randomization, DLD-1 tumor bearing mice were given PO gavage doses of 0 (vehicle), 3, or 10 mg/kg cobimetinib (expressed as free-base equivalents) QD for 21 days. Mice received 25 mg/kg of MEHD7945A via IV bolus injection QW for a total of three injections. In groups that received both agents, cobimetinib was administered first and immediately followed by MEHD7945A.

Administration of cobimetinib at 3 and 10 mg/kg and MEHD7945A at 25 mg/kg resulted in 39%, 62%, and 62% TGI, respectively. However, the combination of at 3 and 10 mg/kg with MEHD7945A at 25 mg/kg resulted in 90% and 108% TGI. The data are shown in FIG. 12A and the study is summarized in FIG. 12B.

### Example 8

### Combination Studies of MEHD7945A with Cobimetinib in Pancreatic BxPC3 Xenograft Model

After randomization, mice were given PO gavage doses of 0 (vehicle), 1, or 5 mg/kg cobimetinib (expressed as free-base equivalents) QD for 21 days. Mice were given 25 mg/kg of MEHD7945A via IV bolus injection QW for a total of three injections. In groups that received both agents, cobimetinib was administered first and immediately followed by MEHD7945A.

Administration of cobimetinib at 1 and 5 mg/kg and MEHD7945A at 25 mg/kg resulted in 88%, 109%, and 107% TGI, respectively. Administration of cobimetinib at 1 or 5 mg/kg with MEHD7945A at 25 mg/kg combined resulted in 113% and 114% TGI, respectively. The data are shown in FIG. 13A and the study is summarized in FIG. 13B. Time to tumor progression (TTP) to twice (2×) initial tumor volume was monitored for each group following 21 days of dosing (see FIG. 13C). In the vehicle-control arm, TTP 2× was 4.5 days. Treatment of mice with the single agent cobimetinib extended TTP 2× to 22 days at 1 mg/kg and 33 days at 5 mg/kg. Treatment of mice with single agent MEHD7945A extended TTP 2× to 39.5 days. Combination of MEHD7945A plus cobimetinib at 1 mg/kg extended TTP 2× to 50.5 days. Likewise, combination of MEHD7945A plus cobimetinib at 5 mg/kg extended TTP 2× to 56 days. A 100% decrease in tumor volume, defined as complete response (CR) was seen in 3 animals in the 5 mg/kg cobimetinib plus MEHD7945A group, but not in any other treatment groups (see FIG. 13C). In FIG. 13 CI=confidence interval; CR=complete response (100% decrease in tumor volume); HB#8=histidine buffer; MCT=0.5% (w/v) methylcellulose, 0.2% (w/v) polysorbate 80; NA=not achieved; PR=partial response (50-99% decrease in tumor volume); TTP=time to tumor progression to twice (2×) or five times (5×) the initial tumor volume represents the group average in days.

All documents cited herein are incorporated by reference. While certain embodiments of invention are described, and many details have been set forth for purposes of illustration, certain of the details can be varied without departing from the basic principles of the invention. Since numerous modifications and changes will be readily apparent to those skilled in the art, it is not desired to limit the invention to the exact construction and process shown as described herein. Accordingly, all suitable modifications and equivalents may be considered to fall within the scope as defined by the claims that follow.

### REFERENCES

Amin D N, Campbell M R, Moasser M M. The role of HER3, the unpretentious member of the HER family, in cancer biology and cancer therapeutics. Sem Cell Dev Biol 2010; 21:944-50.
Bostrom J, Yu S F, Kan D, et al. Variants of the antibody herceptin that interact with HER2 and VEGF at the antigen binding site. Science (New York, N.Y.) 2009; 323:1610-14.
Carracedo A, Ma L, Teruya-Feldstein J, et al. Inhibition of mTORC1 leads to MAPK pathway activation through a PI3K-dependent feedback loop in human cancer. J Clin Invest 2008; 118:3065-74.
Ciardiello F and Tortora G. EGFR antagonists in cancer treatment. N Engl J Med 2008; 358:1160-74.
Diaz L A Jr, Williams R T, Wu J, et al. The molecular evolution of acquired resistance to targeted EGFR blockade in colorectal cancers. Nature 2012; 486(7404):537-40. doi:10.1038/nature11219.
Diep C H, Munoz R M, Choudhary A, et al. Synergistic effect between erlotinib and MEK inhibitors in KRAS wild-type human pancreatic cancer cells. Clin Cancer Res 2011; 17:2744-56.
Downward J. Targeting RAS signalling pathways in cancer therapy. Nat Rev Cancer 2003; 3:11-22. Review.
Eastman A, Perez R P. New targets and challenges in the molecular therapeutics of cancer. Br J Clin Pharmacol 2006; 62:5-14.
Engelman J A, Janne P A, Mermel C, et al. ErbB-3 mediates phosphoinositide 3-kinase activity in gefitinib-sensitive non-small cell lung cancer cell lines. Proceedings of the National Academy of Sciences of the United States of America 2005; 102:3788-93.
Engelman J A, Zejnullahu K, Mitsudomi T, et al. MET amplification leads to gefitinib resistance in lung cancer by activating ERBB3 signaling. Science (New York, N.Y.) 2007; 316:1039-43.
Ferlay J, Steliarova-Foucher E, Lortet-Tieulent, et al. Cancer incidence and mortality patterns in Europe: Estimates for 40 countries in 2012. Eur J. Cancer. 2013; 49:1374-1403.
Frémin C, Meloche S. From basic research to clinical development of MEK1/2 inhibitors for cancer therapy. J Hematol Oncol. 2010; 3: 8. doi:10.1186/1756-8722-3-8.
Harandi A, Zaidi A S, Stoker A M et al. Clinical efficacy and toxicity of anti-EGFR therapy in common cancers. J Oncol 2009:1-14.
Hansen L A, Alexander N, Hogan M E, et al. Genetically null mice reveal a central role for epidermal growth factor receptor in the differentiation of the hair follicle and normal hair development. Am J PathHayes TK and Der CJ. Mutant and wild-type Ras: co-conspirators in cancer. Cancer Discov 2013; 3:24-6.
Holbro T, Beerli R R, Maurer F, et al. The ErbB2/ErbB3 heterodimer functions as an oncogenic unit: ErbB2 requires ErbB3 to drive breast tumor cell proliferation. Proceedings of the National Academy of Sciences of the United States of America 2003a; 100:8933-38.
Holbro T, Civenni, G Hynes N E. The ErbB receptors and their role in cancer progression. Exp Cell Res 2003b; 284:99-110.
Hynes N E and MacDonald G. ErbB receptors and signaling pathways in cancer. Curr Opin Cell Biol 2009; 21:177-84.
Jemal A, Simard E P, Dorell C, et al. Annual report to the Nation on the Status of cancer, 1975-2009, Featuring the burden and trends in human papillomavirus (HPV)-associated cancers and HPV vaccination coverage levels. J Natl Cancer Inst, 2013; 105(3):175-201. doi:10.1093/jnci/djs491
Jean G W, Shah S R. Epidermal growth factor receptor monoclonal antibodies for the treatment of metastatic colorectal cancer. Pharmacotherapy 2008; 28:742-54.
Johnson L, Mercer K, Greenbaum P, et al. Somatic activation of the k-ras oncogene causes early onset lung cancer in mice. Nature 2001; 410:1111-1116.
Johnson G L, Lapadat R. Mitogen-activated protein kinase pathways mediated by ERK, JNK, and p38 protein kinases [review]. Science 2002; 298:1911-12.
Jost M, Huggett T M, Kari C, et al. Epidermal growth factor receptor-dependent control of keratinocyte survival and Bcl-xL expression through a MEK-dependent pathway. J Biol Chem 2001; 276:6320-6.
Jura N, Shan Y, Cao X, et al. Structural analysis of the catalytically inactive kinase domain of the human EGF receptor 3. Proceedings of the National Academy of Sciences of the United States of America 2009; 106:21608-13.
Karapetis C S, Khambata-Ford S, Jonker D J, et al. K-ras mutations and benefit from cetuximab in advanced colorectal cancer. N Engl J Med 2008; 359:1757-65.
Kern F, Niault T, and Baccarini M. Ras and Raf pathways in epidermis development and carcinogenesis. British Journal of Cancer 2011:104; 229-34.
Kitano H. Cancer as a robust system: implications for anticancer therapy. Nat Rev Cancer 2004; 4:227-35.
Menon S S, Whitfield L R, Sadis S, et al. Pharmacokinetics (PK) and pharmacodynamics (PD) of PD 0325901, a second generation MEK inhibitor after multiple oral doses of PD 0325901 to advanced cancer patients. 2005 ASCO Annual Meeting Proceedings.ol 1997; 150:1959-75.
Mirzoeva O K, Das D, Heiser L M, et al. Basal subtype and MAPK/ERK kinase (MEK)-phosphoinositide 3-kinase feedback signaling determine susceptibility of breast cancer cells to MEK inhibition. Cancer Res 2009; 69:565-572.
Misale S, Yaeger R, Hobor S, et al. Emergence of KRAS mutations and acquired resistance to anti-EGFR therapy in colorectal cancer. Nature 2012; 486(7404):532-6. doi:10.1038/nature11156.
Moasser M M. Targeting the function of the HER2 oncogene in human cancer therapeutics. Oncogene 2007; 26:6577-92.
Natarajan M, Lin K-M, Hsueh R C, et al. A global analysis of cross-talk in a mammalian cellular signaling network. Nat Cell Biol 2006; 8:571-80.
Olayioye M A, Neve R M, Lane H A, et al. The ErbB signaling network: receptor heterodimerization in development and cancer. EMBO J. 2000; 19:3159-67.
Pinkas-Kramarski R, Soussan L, Waterman H, et al. Diversification of Neu differentiation factor and epidermal growth factor signaling by combinatorial receptor interactions. EMBO J. 1996; 15:2452-67.
Pinheiro J, Bates D, DebRoy S, et al. nlme: linear and nonlinear mixed effects models. 2009; Version R package version 3.1-92. Riese D J 2nd, van Raaij T M, Plowman G D, et al. The cellular response to neuregulins is governed by complex interactions of the erbB receptor family. Mol Cell Biol 1995; 15:5770-76.
Roberts P J and Der C J. Targeting the RAF-MEK-ERK mitogen-activated protein kinase cascade for the treatment of cancer [review]. Oncogene 2007; 26:3291-310.
Saijo N. What are the reasons for negative phase III trials of molecular-target-based drugs? Cancer Sci 2004; 95:772-6.
Schaefer G, Haber L, Crocker L M, et al. A two-in-one antibody against HER3 and EGFR has superior inhibitory activity compared with monospecific antibodies. Cancer Cell 2011; 20:472-86.
Schoeberl B, West K A, Leszczyniecka M O, et al. MM-121: a human monoclonal antibody ErbB3 antagonist. American Association for Cancer Research 2008. [Poster 7006].
Sergina N V, Moasser M M. The HER family and cancer: emerging molecular mechanisms and therapeutic targets. Trends Mol Med 2007; 13:527-34.
Sergina N V, Rausch M, Wang D, et al. Escape from HER-family tyrosine kinase inhibitor therapy by the kinase-inactive HER3. Nature 2007; 445:437-41.
Shames D S, Carbon J, Walter K, et al. (2013). High heregulin expression is associated with activated HER3 and may define an actionable biomarker in patients with squamous cell carcinomas of the head and neck. PloS One 2013; 8:e56765.
Shi F, Telesco S E, Liu Y, et al. ErbB3/HER3 intracellular domain is competent to bind ATP and catalyze autophosphorylation. Proceedings of the National Academy of Sciences of the United States of America 2010; 107:7692-7.
Sierke S L, Cheng K, Kim H H, et al. Biochemical characterization of the protein tyrosine kinase homology domain of the ErbB3 (HER3) receptor protein. Biochem J 1997; 322(Pt 3):757-63.
Smit V T, Boot A J, Smits A M, et al. KRAS codon 12 mutations occur very frequently in pancreatic adenocarcinomas. Nucleic Acids Res 1988; 16:7773-82.
Sunaga N, Kaira K, Imai H, et al. Oncogenic KRAS-induced epiregulin overexpression contributes to aggressive phenotype and is a promising therapeutic target in non-small-cell lung cancer. Oncogene 2012 Sep. 10. doi: 10.1038/onc.2012.402. [Epub ahead of print]
Sunaga N, Shames D S, Girard L, et al. Knockdown of oncogenic KRAS in non-small cell lung cancers suppresses tumor growth and sensitizes tumor cells to targeted therapy. Mol Cancer Ther. 2011 February; 10(2):336-346.
Threadgill D W, Dlugosz A A, Hansen L A, et al. Targeted disruption of mouse EGF receptor: effect of genetic background on mutant phenotype. Science 1995; 269:230-4.
Troiani T, Martinelli E, Capasso A, et al. Targeting EGFR in pancreatic cancer treatment. Curr Drug Targets 2012; 13:802-10.
Turke A B, Song Y, Costa C, et al. MEK inhibition leads to PI3K/AKT activation by relieving a negative feedback on ERBB receptors. Cancer Res 2012; 72:3228-37.
Wheeler D L, Huang S, Kruser T J, et al. Mechanisms of action acquired resistance to cetuximab: role of HER (ErbB) family members. Oncogene 2008; 27:3944-56.
Wilson T R, Lee D Y, Berry L, et al. Neuregulin-1-mediated autocrine signaling underlies sensitivity to HER2 kinase inhibitors in a subset of human cancers. Cancer Cell 2011; 20:158-72.
Young A, Lou D, McCormick F. Oncogenic and wild-type Ras play divergent roles in the regulation of mitogen-activated protein kinase signaling. Cancer Discovery 2013; 3:112-23.

We claim:

1. A pharmaceutical product comprising (i) GDC-0973 or GDC-0623, or a pharmaceutically acceptable salt thereof; and (ii) MEHD7945A for concurrent or sequential use in the treatment of a hyperproliferative disorder.

2. The pharmaceutical product of claim 1, wherein the hyperproliferative disorder is cancer.

3. The pharmaceutical product of claim 2, wherein the cancer is associated with a KRAS mutation.

4. The pharmaceutical product of claim 2, wherein the cancer is associated with an AKT mutation, overexpression or amplification.

5. The pharmaceutical product of claim 2, wherein the cancer is associated with a PI3K mutation, overexpression or amplification.

6. The pharmaceutical product of claim 2, wherein cancer is selected from, colorectal, mesothelioma, endometrial, pancreatic, breast, lung, ovarian, prostate, melanoma, gastric, colon, renal, head and neck, and glioblastoma.

7. The pharmaceutical product of claim 1, wherein GDC-0973 or a pharmaceutically acceptable salt thereof is administered in combination with MEHD7945A.

8. The pharmaceutical product of claim 1, wherein GDC-0623 or a pharmaceutically acceptable salt thereof is administered in combination with MEHD7945A.

9. The pharmaceutical product of claim 1, wherein GDC-0973 or GDC-0623, or a pharmaceutically acceptable salt thereof is administered simultaneously with MEHD7945A.

10. The pharmaceutical product of claim 1, wherein GDC-0973 or GDC-0623, or a pharmaceutically acceptable salt thereof and MEHD7945A are administered sequentially.

11. A pharmaceutical product comprising (i) GDC-0973 or GDC-0623, or a pharmaceutically acceptable salt thereof; and (ii) MEHD7945A, as a combined preparation for concurrent or sequential use for improving the quality of life of a patient having a hyperproliferative disorder.

12. A kit comprising GDC-0973 or GDC-0623, or a pharmaceutically acceptable salt thereof; and MEHD7945A, a container, and a package insert or label indicating the administration GDC-0068 or GDC-0941, or a pharmaceutically acceptable salt thereof; and MEHD7945A, for treating a hyperproliferative disorder in a patient.

13. A method for treating a hyperproliferative disorder in a patient, comprising administering to the patient a therapeutically effective amount of (i) GDC-0973 or GDC-0623, or a pharmaceutically acceptable salt thereof; and (ii) MEHD7945A.

14. The method of claim 13, wherein the hyperproliferative disorder is cancer.

15. The method of claim 14, wherein the cancer is associated with a KRAS mutation.

16. The method of claim 14, wherein the cancer is associated with AKT mutation, overexpression or amplification.

17. The method of claim 14, wherein the cancer is associated with PI3K mutation, overexpression or amplification.

18. The method of claim 14, wherein cancer is selected from, colorectal, mesothelioma, endometrial, pancreatic, breast, lung, ovarian, prostate, melanoma, gastric, colon, renal, head and neck, and glioblastoma.

19. The method of claim 14, wherein GDC-0973 or a pharmaceutically acceptable salt thereof is administered in combination with MEHD7945A.

20. The method of claim 14, wherein GDC-0623 or a pharmaceutically acceptable salt thereof is administered in combination with MEHD7945A.

21. The method of claim 14, wherein GDC-0973 or GDC-0623, or a pharmaceutically acceptable salt thereof is administered simultaneously with MEHD7945A.

22. The method of claim 14, wherein GDC-0973 or GDC-0623, or a pharmaceutically acceptable salt thereof and MEHD7945A are administered sequentially.
